(19) United States
(12) Patent Application Publication
(10) Pub. No.: US 2010/0048900 A1
(43) Pub. Date: Feb. 25, 2010

(54) AGENT FOR TREATMENT OF VASCULAR LEAKS

(76) Inventors: Thomas Schwarz, Leichlingen (DE); Georg Lentzen, Dortmund (DE)

Correspondence Address: BERLINER & ASSOCIATES, 555 WEST FIFTH STREET, 31ST FLOOR, LOS ANGELES, CA, 90013, US

(21) Appl. No.: 12/516,889

(22) PCT Filed: Dec. 3, 2007
(86) PCT No.: PCT/EP07/10479

§ 371 Date: Sep. 29, 2009

(30) Foreign Application Priority Data
Dec. 1, 2006 (DE) 10 2006 056 766.8

(51) Int. Cl. C07D 239/06

(52) U.S. Cl. 544/335

(57) ABSTRACT

The invention relates to an agent which contains ectoines for the prophylactic or therapeutic treatment of endothelial function disturbances, in particular the VLS.

The invention relates to the use of ectoines for the prophylactic or therapeutic treatment of endothelial function disturbances, for example vascular leaks, as well as a suitable agent.

Low-molecular organic chemical compounds isolated from extremophilic organisms have a remarkable influence on the thermal, chemical and physical stability of complex organic compounds and biological cell structures. Such organic compounds are termed osmolytes or compatible solutes and have meanwhile found their way into numerous cosmetic preparations.

Counting among compatible solutes are sugar, sulfur compounds, polyols, amino acids and amino acid derivatives as well as tetrahydropyrimidine derivatives such as ectoine and hydroxyectoine. They are synthesized from extremophilic microorganisms under stress conditions and serve the purpose of stabilizing the cell structures of these microorganisms under extreme thermal, chemical and physical conditions. An example in this respect are halophile microorganisms that must adapt to the changing salt content in a saline environment and must survive in this environment.

Examples of compatible solutes which have been applied in actual practice are, among others, ectoine and hydroxyectoine.

EP-A-0 553 884 describes purified tetrahydropyrimidine derivatives as well as pharmaceutical compositions containing these derivatives. They are suitable for the protection of the DNA to preclude damage caused by DNA-binding agents, carcinogenic substances, mutagenicity and radiation.

It is furthermore known that ectoine and its derivatives have a stabilizing effect on protein and nucleic acid structures with said effect being conducive to the stabilization of biological material and pharmaceutical preparations.

As stated in WO-00/076528 ectoine and hydroxyectoine are suited to increase the effectiveness of active agents that contain proteins. Protein-containing active agents within this meaning are antibodies and immunotoxins. Especially named in this context is the anti-CD30 immunotoxin Ki-4.dgA.

As elucidated in WO 00/076528 the actual agent is the protein-containing substance. Compatible solutes or ectoines are only used to increase the effectiveness, i.e. as pure adjuvants. Using them for the reduction of cytotoxic activities, in particular the vascular leak syndrome (VLS), is also to be seen with this in mind. In the event of the vascular leak syndrome endothelial cell damage leads, inter alia, to loss of albumin in the intracellular space which causes intrastitial fluid to increasingly accumulate and, primarily, edema, hypotonia and tachycardia to occur.

More often than not the vascular leak syndrome occurs when immunotoxins are liberated under stress conditions, for example in the event of a severe sepsis and septic shock but also when infants or little children are connected to heart-lung machines, in case of burns and the systemic inflammatory response syndrome (SIRS)

Interleukin-2 is an important cytokine which, for example, is therapeutically applied to treat certain forms of cancer. However, the application of this highly effective medical agent is greatly limited due to the toxic side effects it produces. Included here is the vascular leak syndrome which is also encountered with other immunotoxins. The vascular leak syndrome is triggered, for example, by the powerful vegetable toxin ricin.

The vascular leak syndrome, also known as capillary leak syndrome, may also occur during major surgical interventions, for example after bone marrow transplants, cardiopulmonary bypass surgery and hemophagocytic lymphohistiocytosis. Pulmonary edemas arising for instance during an acute lung injury and acute respiratory distress syndrome (ARDS) may as well be accompanied by massive vascular leak problems.

Typical individual symptoms of VLS are, for example, hypertension, hypoalbuminemia, lung edemas, edemas of general nature as well as the vascular leak itself. Similar symptoms are encountered in cases of septic shock, SIRS, hemorrhagic fever types such as dengue fever, Arbovirus fever, Marburg and Ebola infections as well as other tropical fever diseases.

Causes of SIRS may be injuries, burns, major bleeding, ischemia, anaphylaxis as well as hemorrhagic-necrotizing pancreatitis. In this case, aside from a vascular leak significantly reduced or elevated body temperatures, heart rate increases, tachypnea, as the case may be accompanied by hypoxia, and a reduction or increase in leukocytes may occur. This is to be considered a disease pattern similar to sepsis but without an infection being detectable here.

What all the symptoms and disease courses described here have in common is that a disturbance of the endothelial cell barrier occurs that separates the respective body compartments from each other. If this barrier is disturbed by exongenous or endogenous noxae liquid and biomolecules from the vascular system may escape and ingress into the surrounding tissue regions which causes symptoms and consequential disease patterns as mentioned hereinbefore.

The mechanisms leading to increased endothelial cell permeability depend on the mediators. For example, an increase of the IL-2 induced endothelial cell permeability is attributable to the induction of the apoptosis of the endothelial cells. Lipopolysaccharide (LPS), a constituent of the outer membrane of gram-negative bacteria, mediates the increased endothelial cell permeability due to the modulation of interendothelial tight/gap junction associated proteins but also through the induction of an endothelial cell apoptosis. On the other hand, histamine, a very early mediator of the systemic inflammation, causes increased endothelial cell permeability primarily through the modulation of tight/gap junction proteins.

Hydroxyectoine in particular stabilizes the endothelial cell barrier through stabilization of the membrane constituents. Through preferential exclusion hydroxyectoine is excluded from the hydrate envelope of various membrane constituents and thus leads to a compaction of the proteins and other membrane components (lipids etc.). For noxae it is now more difficult to reach them (receptor level, e.g. IL-2 receptor) and the downstream signaling pathways are activated less powerfully, an induction of apoptosis can be prevented.

This compaction as well stabilizes the membrane constituents (protein complexes) forming the light junctions (connections between the endothelial cells). These can thus be influenced to a lesser degree by LPS or histamine present in the vascular system so that the junctions between the cells are maintained to a greater extent. In this way, the endothelial cell barrier is stabilized in the event of is LPS/histamine impairments as well.

Accordingly, ectoines are suited both for the protection against and treatment of a vasculitis and in particular of an endothelial dysfunction or functional disturbances of the endothelium, hereinafter usually termed vascular leak or VLS. Moreover, its effectiveness also covers functional disturbances of the epithelial cell barrier function.

Investigations have shown that ectoines and in particular ectoine itself and hydroxyectoine may be used as sole active agents to counteract vascular leaks. Therefore, these substances are suited for prophylaxis and treatment of the VLS.

Accordingly, the invention relates to an agent for prophylaxis and inhibition or treatment of vascular leaks. In said agent ectoines in particular as sole active substances are used.

For the purposes of the invention ectoines are (4S)-1,4,5,6-tetrahydropyrimidine-4-carboxylic acid and its derivatives, especially their pharmaceutically acceptable ester, salts and amides. The tetrahydropyrimidine carboxylic acids may have a lower alkyl group at 2-position, for example a C₁₋₅ alkyl group, in particular a methyl group. At 5-position the tetrahydropyrimidine may be substituted by a hydroxy group, in particular by a (5S) hydroxy group. The hydroxy group may be etherified or esterified so as to be pharmaceutically acceptable.

By disturbances of the endothelial functions all forms of vascular leaks and dysfunctions are understood as they have been described hereinbefore in the context of diseases and symptoms of illness dealt with in more detail. It is understood that the symptoms of illness must not encompass or reflect the entire disease pattern; the agent according to the invention may also be employed for prophylaxis or treatment of individual symptoms which arise in connection with endothelial function disturbances.

Preferred ectoines are ectoines themselves, (4S)-2-methyl-1,4,5,6-tetrahydropyrimidine-4-carboxylic acid and hydroxyoctoine, (4S,5S)-5-hydroxy-2-methyl-1,4,5,6-tetrahydropyrimidine-4-carboxylic acid.

The invention proposes that several ectoines may be applied together. As derivatives those shall be considered that essentially have the same or better effects on VLS than the relevant parent substance.

In the agent proposed by the invention the ectoine may of course be combined with the customary adjuvants and auxiliary substances. Said agent may be provided in the form of tablets, capsule or as solution for oral or parenteral administration, preferably in the form of an aqueous injection solution. It may be applied in doses ranging between 10 and 10 000 mg, preferably between 20 and 1000 mg and in particular between 50 and 500 mg,

As proposed by the invention the ectoines to be used, in particular ectoine and hydroxyectoine, may be combined with each other.

The agent according to the invention relates to any form of the vascular leak syndrome, no matter which circumstances have triggered said syndrome. It relates in particular to those forms of vascular leaks that have been caused by toxins and medical drugs and preparations, for example vascular leaks caused through the administration of interleukin-2. However, ectoines are as well suited to counteract the effect of vegetable toxins, such as ricin, in the event these trigger the VLS. Another field of application is postoperative prophylaxis aimed at preventing a vascular leak syndrome, for example in the context of the above named indications, and the prophylaxis and treatment of the VLS, in particular with infants and little children treated with the help of heart-lung machinery, as well as with hypoxia.

Within the scope of the invention ectoines may be used in the event of the retinoic acid syndrome. When treating acute promyelocytic leukaemia with all-trans retinoic acid (ATRA Therapy) very severe side effects are occasionally encountered in the form of quite a number symptoms such as fever, gain in weight, interstitial pulmonary infiltrates (VLS), pleural and pericardial effusion, episodic high blood pressure and acute kidney failure, cf. R. S. Larson and M. S. Talman, Best Pract. Res. Clin. Haematol. 2003 September; 16 (3):453-61.

Another field of application is the capillary leak syndrome during the acitretin therapy of psoriasis or the occurrence of the acute respiratory distress syndrome (ARDS) in the event of psoriasis. The treatment of psoriasis using retinoic acid may, in rare cases, trigger the VLS with life-threatening effects arising, cf. M. H. Vermeer and S. Pavel, J Am. Acad. Dermatol. 2006 Oct. 20.

With pustular and erythrodermic psoriasis a complication may occasionally be encountered in the form of the acute respiratory distress syndrome (ARDS) which can be traced back to the capillary leak syndrome in the lung, cf. J. S. Sadeh at al., Arch. Dermatol. 1997 June, 133 (6):747-50.

It is assumed that the protective effects of ectoines are linked with a stabilization of the endothelial cells, i.e. the active agents counteract a vasculitis caused by disease or treatment. There are indications that ectoines act via the cell membranes and have a positive effect on inflammatory phenomena.

The effects of ectoines claimed within the scope of this invention are elucidated in more detail by the following examples.

### EXAMPLE 1

In-vitro examinations on the inhibition of the VLS induced by interleukin-2.

Using human endothelial cells (HUVEC cells) the inhibition of the IL-2 induced VLS through ectoine and hydroxyectoine was investigated in an in-vitro system. In this context the concentrations of Proleukin® (by Chiron) were determined that lead to the spherical deformation of the HUVEC cells. The spherical deformation of HUVEC cells is considered as a sign for the triggering of the VLS, cf. Baluna et al., PNAS 3957-3962, March 1999. Same as VLS, this phenotype visible under the light microscope is based on the bonding of membrane proteins, the integrins.

The cells were incubated with Proleukin solely or in combination with ectoine and hydroxyectoine. To verify the specificity of the compatible solutes also trehalose and glucose were employed additionally.

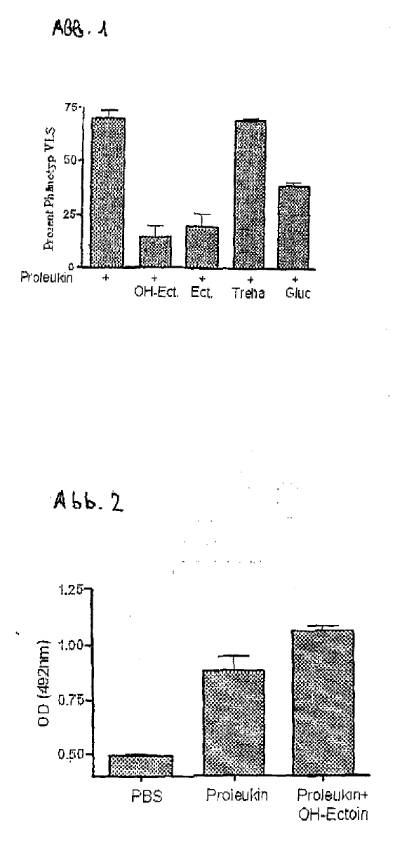

After 4 to 6 days the proportion of normal and spherically deformed cells of a visual field are determined by microscopic assessment. The results are summarized in FIG. 1.

FIG. 1 shows the VLS inhibition percentage on the basis of HUVEC cells after application of 2 mM of Proleukin solely or in combination with 20 mM of hydroxyectoine (OH-Ect.), ectoine (Ect.), trehalose (Treha) and glucose (Gluc).

Both hydroxyectoine and ectoine are capable of significantly reduce the VLS phenotype. A protective effect, though of slightly less significance, is also noticeable in the case of glucose, however not with trehalose.

### EXAMPLE 2

Effects of hydroxyectoine on the Proleukin-induced interferon-γ-synthesis of human lymphocytes.

interleukin-2, IL-2, same as Proleukin leads to the activation of immune effector cells which can be determined, inter alia, by an increased interferon-γ secretion of NK cells and T-lymphocytes.

For this purpose NK cells and T cells of healthy-blood donors were isolated by means of the MACS technique (Magnetic Cell Sorting, Milteniy). The of detection of the cells expressing the surface markers CE 56, NKG2D and CD 3 was brought about by FACS analyses.

The cells without further additives have been incubated for 48 hours with 250 U Proleukin or with 250 U Proleukin in combination with 20 mM hydroxyectoine. Following this, the concentration of interferon-γ was determined in the supernatant of the cells with the aid of ELISA (triple determinations from two independent experiments). The results have shown that hydroxyectoine does not inhibit the induction of the interferon-γ synthesis, on the contrary slightly increased values were found (see FIG. 2).

The in-vitro data have shown that the tested compatible solutes bring about a reduction of the Proleukin-induced VLS phenotype of HUVEC cells, with this not being connected with a reduction of the activity relating to the interferon induction of primary human effector cells.

1. Agent for the prophylactic or therapeutic treatment of endothelial dysfunctions, said agent containing at least one ectoine or a pharmaceutically acceptable derivative thereof.

2. Agent according to claim 1 for the treatment of vascular leaks.

3. Agent according to claim 1 or 2 for the treatment of vasculitis and in particular of the VLS, SIRS, ARDS.

4. Agent according to any one of the above claims for prophylaxis or treatment of vascular leaks caused by toxins or medical preparations or drugs.

5. Agent according to claim 4 for the inhibition of VLS induced by Interleukin-2.

6. Agent according to claim 1 or 2 for the treatment of VLS triggered by hemorrhagic fevers.

7. Agent according to any one of the above claims, characterized in that it contains an ectoine as sole active substance/sole active substances.

8. Agent according to any one of the above claims in a form conducive to oral or parenteral administration to a patient.

9. Agent according to claim 8 in the form of an aqueous injection solution.

10. Agent according to claim 8 or 9, characterized in that it contains at least one ectoine in an amount ranging between 10 and 10000, preferably between 20 and 1000 mg and especially preferred between 50 and 500 mg.
